(19) United States
(12) Patent Application Publication
(10) Pub. No.: US 2022/0375580 A1
(43) Pub. Date: Nov. 24, 2022

(54) System and Method for Obtaining and Analyzing Ultrasound Data

(71) Applicant: Good Type, Inc. d/b/a BB Imaging, Austin, TX (US)

(72) Inventors: Blanca Lesmes, Austin, TX (US); Benedict Buentipo, Austin, TX (US)

(21) Appl. No.: 17/749,871

(22) Filed: May 20, 2022

Related U.S. Application Data
(60) Provisional application No. 63/190,926, filed on May 20, 2021.

(51) Int. Cl. G16H 40/20; G16H 15/00; G16H 50/20; G16H 30/40

(52) U.S. Cl. CPC G16H 40/20; G16H 15/00; G16H 50/20; G16H 30/40

(57) ABSTRACT

A system and computerized method for obtaining and analyzing ultrasound data includes creating by a computer, an electronic ultrasound order for a subject, creating by a computer, an electronic worklist, and downloading the electronic worklist from the computer to an ultrasound machine, wherein the electronic worklist comprises instructions to conduct one or more sets of scanning sweeps, and the ultrasound machine is remotely located with respect to the computer, receiving by the computer, one or more computer files containing the ultrasound data corresponding to the one or more sets of scanning sweeps from the ultrasound machine, conducting by the computer, diagnostics and an evaluation for the subject using the set of scanning sweeps, and creating by the computer, an electronic report for the subject using the diagnostics and the evaluation.

### CROSS-REFERENCE TO RELATED APPLICATIONS

This application claims priority to and is a non-provisional patent application of U.S. provisional patent application Ser. No. 63/190,926 filed on May 20, 2021, which is hereby incorporated by reference in its entirety.

### STATEMENT OF FEDERALLY FUNDED RESEARCH

Not applicable.

### TECHNICAL FIELD OF THE INVENTION

The present invention relates in general to medical technology. In particular, the present invention relates to a system and method for obtaining and analyzing ultrasound data.

### BACKGROUND OF THE INVENTION

Rural communities do not have access to specialists and diagnostics that can help identify pathology in high-risk pregnancies (The American College of Obstetrics and Gynecology, 2014). Current ultrasound technology makes the acquisition, recognition, and diagnosis of images difficult and highly dependent on the availability of competent sonographers and providers (Community for Accredited Online Schools, 2019). These technicians are the linchpins to providing care and capturing the images critical to determining a correct diagnosis in prenatal care (Karim J N, Salomon, Papageorghiou, & Roberts, 2017).

The scarcity of sonography resources results in underutilization of billing, system leakage, and organizational inefficiencies. More importantly, unidentified risks in pregnancies lead to lack of early intervention during and post-pregnancy, lack of preparation for care teams at birth, and increased rates of morbidity and mortality (Reinsurance Group of America, 2019). In brief, the problem is that maternal mortality in the US is among the highest of any industrialized nation. (Martin & Montagne, 2017).

Moreover, research indicates that unskilled sonographers or under-skilled healthcare providers lead to worse outcomes in pregnancy. Additionally, women in rural communities are 9% more likely to have a dangerous birth experience because of a lack of access to care. Even state-level studies have linked distance to care with adverse health outcomes. For example, drive time was associated with premature delivery in a study of women in Georgia. Those required to drive 45 minutes or more to their delivery hospital were 1.53 times more likely to have a premature delivery than women who have to drive less than 15 minutes.

Improvements to screening and higher detection of at-risk pregnancy with ultrasound can improve maternal morbidity and mortality by as much as 35%. As a result, there is a need for a system and method for obtaining and analyzing ultrasound data.

### SUMMARY OF THE INVENTION

Embodiments of the present disclosure transform the delivery of ultrasound, which has been essentially unchanged since the 1980s, by reducing the dependency on skilled technicians to extend prenatal care services to the most vulnerable patients. These experienced technicians are scarce and hard to recruit and retain. For this reason, embodiments of the present disclosure decouple the reliance on an “in-person” skilled sonographer to have better outcomes.

One embodiment of the present disclosure provides a computerized method for obtaining and analyzing ultrasound data. The method includes creating by a computer, an electronic ultrasound order for a subject; creating by a computer, an electronic worklist, and downloading the electronic worklist from the computer to an ultrasound machine, wherein the electronic worklist comprises instructions to conduct one or more sets of scanning sweeps, and the ultrasound machine is remotely located with respect to the computer; receiving by the computer, one or more computer files containing the ultrasound data corresponding to the one or more sets of scanning sweeps from the ultrasound machine; conducting by the computer, diagnostics and an evaluation for the subject using the set of scanning sweeps; and creating by the computer, an electronic report for the subject using the diagnostics and the evaluation.

In one aspect, the method further includes conducting the one or more sets of scanning sweeps using a transducer connected to the ultrasound machine. In another aspect, the one or more sets of scanning sweeps are conducted by a technician, the subject or an automated device. In another aspect, the one or more set scanning sweeps include: a set of up and down scanning sweeps; a set of left and right scanning sweeps; a set of lower right to upper left scanning sweeps; or a set of lower left to upper right scanning sweeps. In another aspect, the instructions comprise an electronic graphical representation of the one or more sets of scanning sweeps. In another aspect, the method further includes reviewing the set of scanning sweeps. In another aspect, the method includes sending, by the computer, an acceptance or rejection of all or part of the set of scanning sweeps to a user device. In another aspect, the acceptance or rejection is provided in real time or near real time. In another aspect, the review, acceptance or rejection is performed by an artificial intelligence or machine learning program running on the computer. In another aspect, the rejection include one or more instructions on how to overcome the rejection. In another aspect, the method further includes: receiving by the computer, one or more computer files containing a new set of scanning sweeps corresponding the rejected scanning sweeps; reviewing by the computer, the new set of scanning sweeps; and sending by the computer, an acceptance or rejection of all or part of the new set of scanning sweeps to user device. In another aspect, the method further includes providing by the computer, an electronic diagnostic tool comprising a set of diagnostic data selections and an interactive display of the one or more sets of scanning sweeps. In another aspect, the method further includes performing by the computer, one or more measurements or percentile calculations. In another aspect, the one or more measurements or percentile calculations are automatically performed by the computer. In another aspect, the method further includes providing by the computer, a diagnosis based on the diagnostics, the evaluation, a patient survey or a percentile calculations using an artificial intelligence or machine learning program running on the computer. In another aspect, the method further includes annotating by the computer, the one or more computer files. In another aspect, the method further includes prioritizing by the computer, a set of images corresponding to the one or more sets of scanning sweeps. In another aspect, the method further includes incorporating by the computer, the prioritized set of images into the electronic report. In another aspect, the method further includes creating by the computer, a virtual reality or augmented reality of one or more images corresponding to the one or more sets of scanning sweeps. In another aspect, the method further includes creating by the computer, a non fungible token of one or more images corresponding to the one or more sets of scans. In another aspect, the method further includes providing by the computer, an electronic report generation tool comprising a report template and a set of reporting selections corresponding to one or more portions of the subject. In another aspect, the method further includes sending by the computer, the electronic report to one or more electronic devices corresponding to the subject, a medical professional or both. In another aspect, the method further includes establishing by the computer, a communications link between the computer and the ultrasound machine.

Another embodiment of the present disclosure provides a system for obtaining and analyzing ultrasound data. The system includes an ultrasound machine, one or more user devices, and a computer communicably coupled to the ultrasound machine and the one or more user devices. The computer is remotely located with respect to the ultrasound machine and the computer is configured to: create an electronic ultrasound order for a subject, create and download an electronic worklist to the ultrasound machine, wherein the electronic worklist comprises instructions to conduct one or more sets of scanning sweeps, receive one or more computer files containing the ultrasound data comprising the one or more sets of scanning sweeps from the ultrasound machine, conduct diagnostics and an evaluation for the subject using the set of scanning sweeps, and create an electronic report for the subject using the diagnostics and the evaluation.

In one aspect, the one or more sets of scanning sweeps are conducted by a technician, the subject or an automated device. In another aspect, the one or more set scanning sweeps includes: a set of up and down scanning sweeps; a set of left and right scanning sweeps; a set of lower left to upper right scanning sweeps; or a set of lower left to upper right scanning sweeps. In another aspect, the instructions comprise an electronic graphical representation of the one or more sets of scanning sweeps. In another aspect, the computer is further configured to review the set of scanning sweeps. In another aspect, the computer is further configured to send an acceptance or rejection of all or part of the set of scanning sweeps to the computer. In another aspect, the acceptance or rejection is provided in real time or near real time. In another aspect, the review, acceptance or rejection is performed by an artificial intelligence or machine learning program running on the computer. In another aspect, the rejection include one or more instructions on how to overcome the rejection. In another aspect, the computer is further configured to: receive one or more computer files containing a new set of scanning sweeps corresponding the rejected scanning sweeps; review the new set of scanning sweeps; and send an acceptance or rejection of all or part of the new set of scanning sweeps to the ultrasound machine. In another aspect, the computer is further configured to provide an electronic diagnostic tool comprising a set of diagnostic data selections and an interactive display of the one or more sets of scanning sweeps. In another aspect, the computer is further configured to perform one or more measurements or percentile calculations. In another aspect, the one or more measurements or percentile calculations are automatically performed by the computer. In another aspect, the computer is further configured to provide a diagnosis based on the diagnostics, the evaluation, a patient survey or a percentile calculations using an artificial intelligence or machine learning program running on the computer. In another aspect, the computer is further configured to annotate the one or more computer files. In another aspect, the computer is further configured to prioritize a set of images corresponding to the one or more sets of scanning sweeps. In another aspect, the computer is further configured to incorporate the prioritized set of images into the electronic report. In another aspect, the computer is further configured to create a virtual reality or augmented reality of one or more images corresponding to the one or more sets of scanning sweeps. In another aspect, the computer is further configured to create a non fungible token of one or more images corresponding to the one or more sets of scans. In another aspect, the computer is further configured to provide an electronic report generation tool comprising a report template and a set of reporting selections corresponding to one or more portions of the subject. In another aspect, the computer is further configured to send the electronic report to one or more electronic devices corresponding to the subject, a medical professional or both.

### BRIEF DESCRIPTION OF THE DRAWINGS

For a more complete understanding of the features and advantages of the present invention, reference is now made to the detailed description of the invention along with the accompanying figures, in which:

FIG. 1 depicts an illustrative process flow for clients in accordance with one embodiment of the present disclosure;

FIG. 2 depicts a system for obtaining and analyzing ultrasound data in accordance with one embodiment of the present disclosure;

FIG. 3 depicts a flow chart of a method for obtaining and analyzing ultrasound data in accordance with one embodiment of the present disclosure;

FIG. 4 depicts a flow chart of a method for obtaining and analyzing ultrasound data in accordance with another embodiment of the present disclosure;

FIGS. 5A-5D depict various patient survey screens in accordance with one embodiment of the present disclosure;

FIG. 6 depicts an instruction screen with feedback from remote sonographer in accordance with one embodiment of the present disclosure;

FIG. 7 depicts a remote sonographer diagnostics screen in accordance with one embodiment of the present disclosure;

FIG. 8 depicts a sonographer diagnostic tools screen in accordance with one embodiment of the present disclosure;

FIGS. 9A-9C depict a patient report in accordance with one embodiment of the present disclosure; and

FIG. 10 depicts a flow chart of a method for obtaining and analyzing ultrasound data in accordance with another embodiment of the present disclosure.

### DETAILED DESCRIPTION OF THE INVENTION

Illustrative embodiments of the system of the present application are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

Various embodiments of the present disclosure, also referred to TeleScan™, allow an unskilled medical professional, such as entry-level sonographers, clinicians or medical assistants who are not formally trained in ultrasound (hereinafter referred to as “drivers” or “users”), to utilize existing FDA approved ultrasound machine (such as GE Voluson E6 510k K192159, GE Voluson S8 510k K180374, and Philips EPIQ 510k K182857) to successfully acquire video clips of a fetus and upload them to a computer via a network or the “cloud.” “Remote” highly skilled professional sonographers, artificial intelligence or a machine learning program then view images and successfully complete a diagnostic report. This is done by the “slicing” of video clips into singular images. The system ensures accurate measurement transfer necessary for detection and diagnosis of medical anomalies. Additionally, the software allows for physician interpretation and submission of reports to referring providers. Notably, the embodiments described herein are not limited pregnancy-related sonograms, but are applicable to sonograms of any portion of a patient's body.

To begin, the driver creates an order in TeleScan™. This order serves as a request to start a new patient interaction. The TeleScan™ software then communicates with the ultrasound machine to create an ultrasound study. This order serves as a request to start a new patient interaction. The driver then uses the ultrasound machine to capture transabdominal scans. Ultrasound is transmitted in a unique international standard called DICOM. These DICOM files are then uploaded to TeleScan™'s HIPAA-compliant secure cloud.

TeleScan™ has three main functions. First, Sonographers can review ultrasound images remotely and prepare diagnostic reports. Secondly, physicians can review and finalize ultrasound reports. Third, TeleScan™ supports entry-level sonographers, clinicians, or untrained medical assistants to obtain video clips for remote reviewing.

SaaS must comply with all digital health and related data laws, including but not limited to HIPAA. TeleScan™ meets these requirements.

Multiple workshops have been performed with volunteer patients, drivers, and sonographers to ensure the efficacy of the innovation.

Various non-limiting embodiments will now be described in more detail.

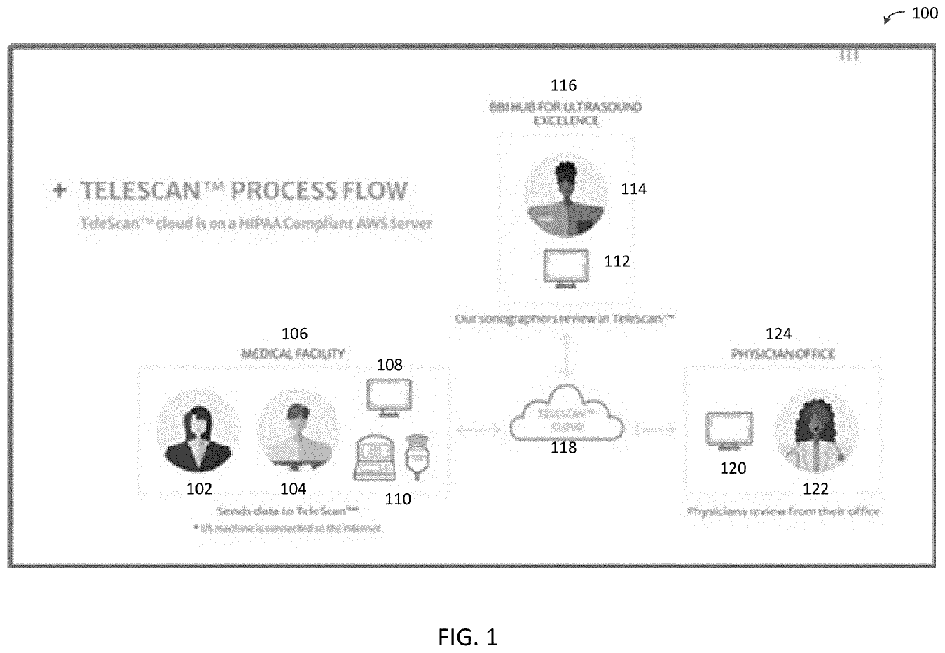

Now referring to FIG. 1, an illustrative process flow 100 for clients in accordance with one embodiment of the present disclosure is shown. A medical assistant or “driver” 102 attending to the patient 104 in-person at a medical facility 106 gathers patient demographic information and patient history. After gathering the patient data, the medical assistant 102 uses the an embodiment of the present disclosure accessible via their client device 108 to guide them in acquiring short ultrasound video clips (sweeps), using their facility's existing ultrasound equipment 110.

The videos of the ultrasound scan automatically upload to a computer 112 or other suitable device of a highly-skilled sonographer 114 at a remote location 116 via the “cloud” 118 or other suitable communication link. The highly-skilled sonographer 114 will provide immediate feedback on video quality and prompt the medical assistant 102 to redo any “sweeps” that were subpar. In some cases, the feedback is provided by a computer, either computer 112 or a other computer communicably coupled to the cloud 118, running an artificial intelligence or machine learning program.

Once all videos are uploaded, the remote sonographer 114 takes over the process. The sonographer 114 reviews the patient data, then conducts their examination of the video clips. They will proceed to “slice” the video into singular images on which they can measure anatomical structures. The measurements annotated on the images will flow into the patient report. The sonographer 114 completes the report with their impressions and forwards the information to a client device 120 of the referring physician 122 at the physician's office 124 for interpretation and patient follow up in clinic or via telemedicine.

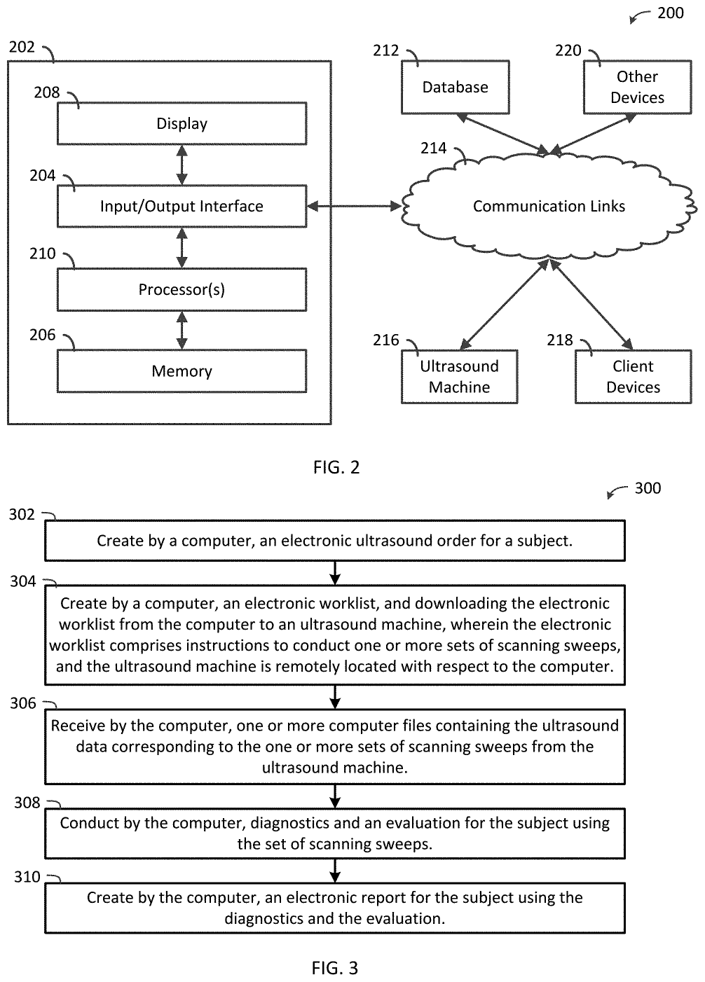

Referring now to FIG. 2, a system 200 for obtaining and analyzing ultrasound data in accordance with one embodiment of the present disclosure is shown. An computer 202 includes an input/output interface 204, a memory 206, a display 208 communicably coupled to the input/output interface 204, and one or more processors 810 communicably coupled to the input/output interface 204 and the memory 206. Note that the computer 202 may include more or less components than specifically described herein. Moreover, the computer 202 can be any type of processing device capable of performing the functions described herein. The memory 206 can be local, remote or distributed. Likewise, the one or more processors 210 can be local, remote or distributed. Moreover, the computer 202 may include database 212 or be communicably coupled to the database 212 via one or more communication links 214. As a result, the database 212 can be local, remote or distributed. The input/output interface 204 can be any mechanism for facilitating the input and/or output of information (e.g., web-based interface, touchscreen, keyboard, mouse, display, printer, etc.) Moreover, the input/output interface 204 can be a remote device communicably coupled to the one or more processors 210 via one or more communication links 214 (e.g., network(s), cable(s), wireless, satellite, etc.). The one or more communication links 214 can communicably couple the computer 202 to one or more ultrasound machines 216, client or user devices 218 and other devices 220 (e.g., databases, remote devices, etc.). The client or user devices 218 can be other computers, laptops, tablets, hand held communication devices, an interface to the ultrasound machine 216 or any other suitable device.

As described above, the system 200 includes an ultrasound machine 216, one or more client or user devices 218, and a computer 200 communicably coupled to the ultrasound machine 216 and the one or more client or user devices 218. The computer 202 is remotely located with respect to the ultrasound machine 216. Moreover, the computer 202 is configured to: create an electronic ultrasound order for a subject, create and download an electronic worklist to the ultrasound machine 216, wherein the electronic worklist comprises instructions to conduct one or more sets of scanning sweeps, receive one or more computer files containing the ultrasound data comprising the one or more sets of scanning sweeps from the ultrasound machine 216, conduct diagnostics and an evaluation for the subject using the set of scanning sweeps, and create an electronic report for the subject using the diagnostics and the evaluation.

In one aspect, the one or more sets of scanning sweeps are conducted by a technician, the subject or an automated device. In another aspect, the one or more set scanning sweeps includes: a set of up and down scanning sweeps; a set of left and right scanning sweeps; a set of lower left to upper right scanning sweeps; or a set of lower left to upper right scanning sweeps. In another aspect, the instructions comprise an electronic graphical representation of the one or more sets of scanning sweeps. In another aspect, the computer is further configured to review the set of scanning sweeps. In another aspect, the computer is further configured to send an acceptance or rejection of all or part of the set of scanning sweeps to the computer. In another aspect, the acceptance or rejection is provided in real time or near real time. In another aspect, the review, acceptance or rejection is performed by an artificial intelligence or machine learning program running on the computer. In another aspect, the rejection include one or more instructions on how to overcome the rejection. In another aspect, the computer is further configured to: receive one or more computer files containing a new set of scanning sweeps corresponding the rejected scanning sweeps; review the new set of scanning sweeps; and send an acceptance or rejection of all or part of the new set of scanning sweeps to the ultrasound machine. In another aspect, the computer is further configured to provide an electronic diagnostic tool comprising a set of diagnostic data selections and an interactive display of the one or more sets of scanning sweeps. In another aspect, the computer is further configured to perform one or more measurements or percentile calculations. In another aspect, the one or more measurements or percentile calculations are automatically performed by the computer. In another aspect, the computer is further configured to provide a diagnosis based on the diagnostics, the evaluation, a patient survey or a percentile calculations using an artificial intelligence or machine learning program running on the computer. In another aspect, the computer is further configured to annotate the one or more computer files. In another aspect, the computer is further configured to prioritize a set of images corresponding to the one or more sets of scanning sweeps. In another aspect, the computer is further configured to incorporate the prioritized set of images into the electronic report. In another aspect, the computer is further configured to create a virtual reality or augmented reality of one or more images corresponding to the one or more sets of scanning sweeps. In another aspect, the computer is further configured to create a non fungible token of one or more images corresponding to the one or more sets of scans. In another aspect, the computer is further configured to provide an electronic report generation tool comprising a report template and a set of reporting selections corresponding to one or more portions of the subject. In another aspect, the computer is further configured to send the electronic report to one or more electronic devices corresponding to the subject, a medical professional or both.

Now referring to FIG. 3, a flow chart of a method 300 for obtaining and analyzing ultrasound data in accordance with one embodiment of the present disclosure is shown. The method includes: creating by a computer, an electronic ultrasound order for a subject in block 302; creating by a computer, an electronic worklist, and downloading the electronic worklist from the computer to an ultrasound machine in block 304, wherein the electronic worklist comprises instructions to conduct one or more sets of scanning sweeps, and the ultrasound machine is remotely located with respect to the computer; receiving by the computer, one or more computer files containing the ultrasound data corresponding to the one or more sets of scanning sweeps from the ultrasound machine in block 306; conducting by the computer, diagnostics and an evaluation for the subject using the set of scanning sweeps in block 308; and creating by the computer, an electronic report for the subject using the diagnostics and the evaluation in block 310.

In one aspect, the method further includes conducting the one or more sets of scanning sweeps using a transducer connected to the ultrasound machine. In another aspect, the one or more sets of scanning sweeps are conducted by a technician, the subject or an automated device. In another aspect, the one or more set scanning sweeps include: a set of up and down scanning sweeps; a set of left and right scanning sweeps; a set of lower right to upper left scanning sweeps; or a set of lower left to upper right scanning sweeps. In another aspect, the instructions comprise an electronic graphical representation of the one or more sets of scanning sweeps. In another aspect, the method further includes reviewing the set of scanning sweeps. In another aspect, the method includes sending, by the computer, an acceptance or rejection of all or part of the set of scanning sweeps to a user device. In another aspect, the acceptance or rejection is provided in real time or near real time. In another aspect, the review, acceptance or rejection is performed by an artificial intelligence or machine learning program running on the computer. In another aspect, the rejection include one or more instructions on how to overcome the rejection. In another aspect, the method further includes: receiving by the computer, one or more computer files containing a new set of scanning sweeps corresponding the rejected scanning sweeps; reviewing by the computer, the new set of scanning sweeps; and sending by the computer, an acceptance or rejection of all or part of the new set of scanning sweeps to user device. In another aspect, the method further includes providing by the computer, an electronic diagnostic tool comprising a set of diagnostic data selections and an interactive display of the one or more sets of scanning sweeps. In another aspect, the method further includes performing by the computer, one or more measurements or percentile calculations. In another aspect, the one or more measurements or percentile calculations are automatically performed by the computer. In another aspect, the method further includes providing by the computer, a diagnosis based on the diagnostics, the evaluation, a patient survey or a percentile calculations using an artificial intelligence or machine learning program running on the computer. In another aspect, the method further includes annotating by the computer, the one or more computer files. In another aspect, the method further includes prioritizing by the computer, a set of images corresponding to the one or more sets of scanning sweeps. In another aspect, the method further includes incorporating by the computer, the prioritized set of images into the electronic report. In another aspect, the method further includes creating by the computer, a virtual reality or augmented reality of one or more images corresponding to the one or more sets of scanning sweeps. In another aspect, the method further includes creating by the computer, a non fungible token of one or more images corresponding to the one or more sets of scans. In another aspect, the method further includes providing by the computer, an electronic report generation tool comprising a report template and a set of reporting selections corresponding to one or more portions of the subject. In another aspect, the method further includes sending by the computer, the electronic report to one or more electronic devices corresponding to the subject, a medical professional or both. In another aspect, the method further includes establishing by the computer, a communications link between the computer and the ultrasound machine.

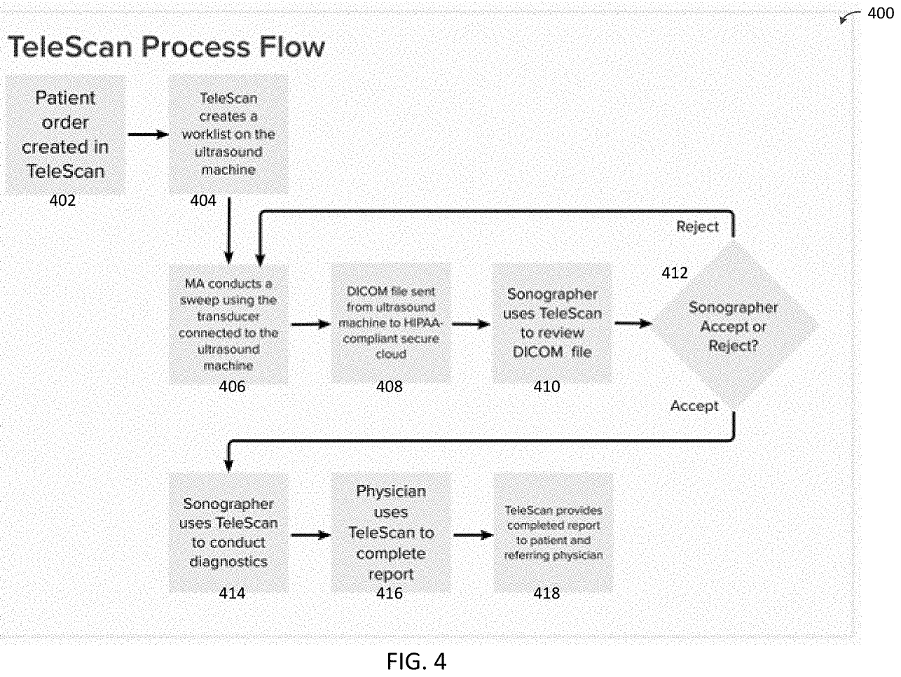

Referring now to FIG. 4, a flow chart of a method 400 for obtaining and analyzing ultrasound data in accordance with another embodiment of the present disclosure is shown. A patient order is created in TeleScan™ in block 402, and TeleScan™ creates a worklist on the ultrasound machine. The medical assistant conducts a sweep using the transducer connected to the ultrasound machine in block 406. The DICOM file is sent from the ultrasound machine to a HIPAA-compliant secure cloud in block 408. The sonographer uses TeleScan™ to review the DICOM file. If the sonographer rejects the DICOM file, as determined in decision block 412, the process returns to block 406 where the medical assistant re-conducts the sweep. If the sonographer accepts the DICOM file, as determined in decision block 412, the sonographer uses TeleScan™ to conduct diagnostics in block 414. The physician then uses TeleScan™ to complete the report in block 416, and TeleScan™ provides the completed report to the patient and the referring physician in block 418.

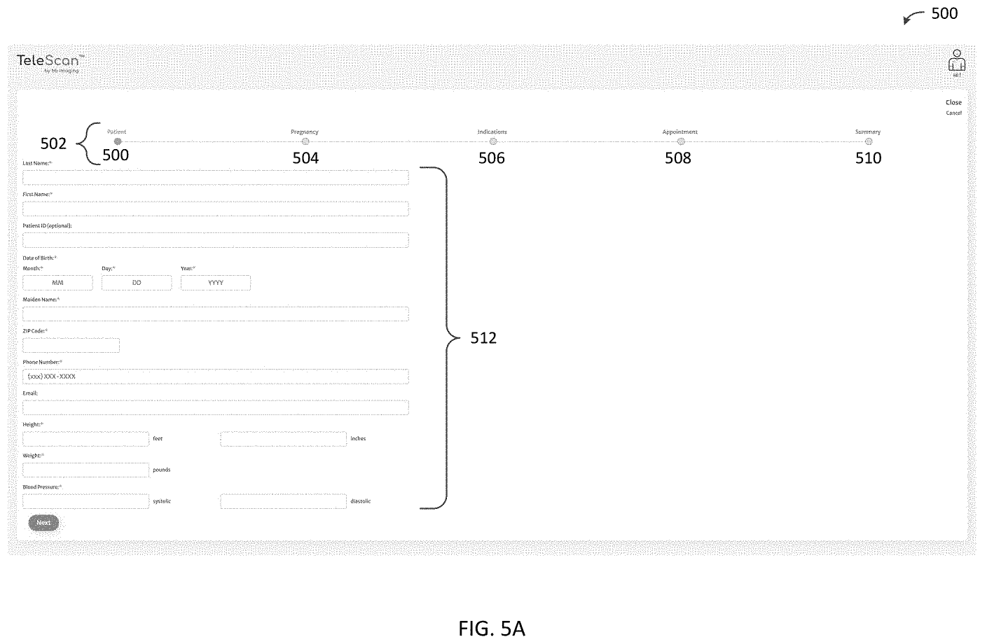

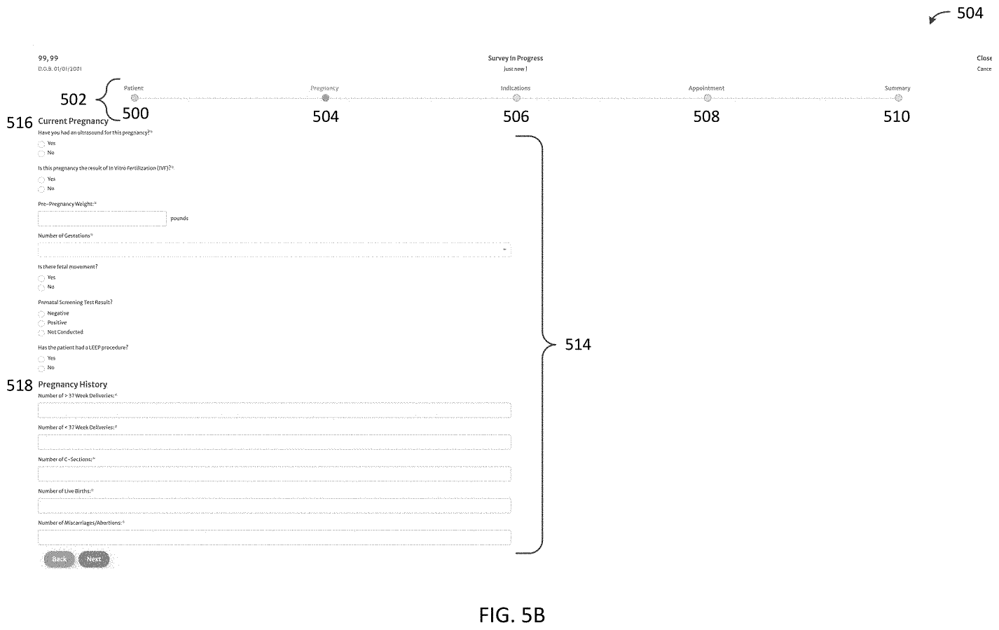

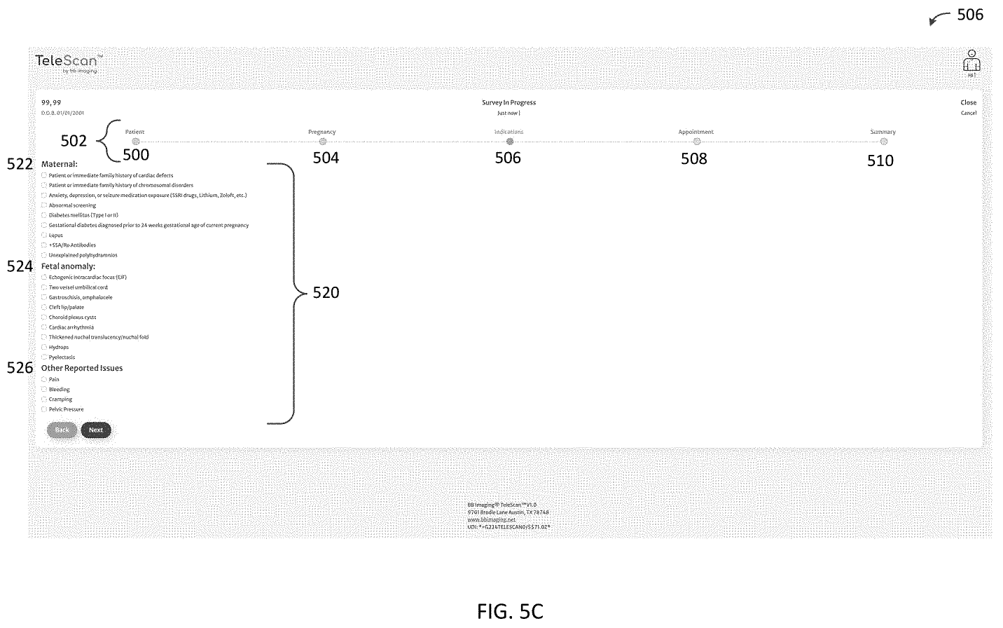

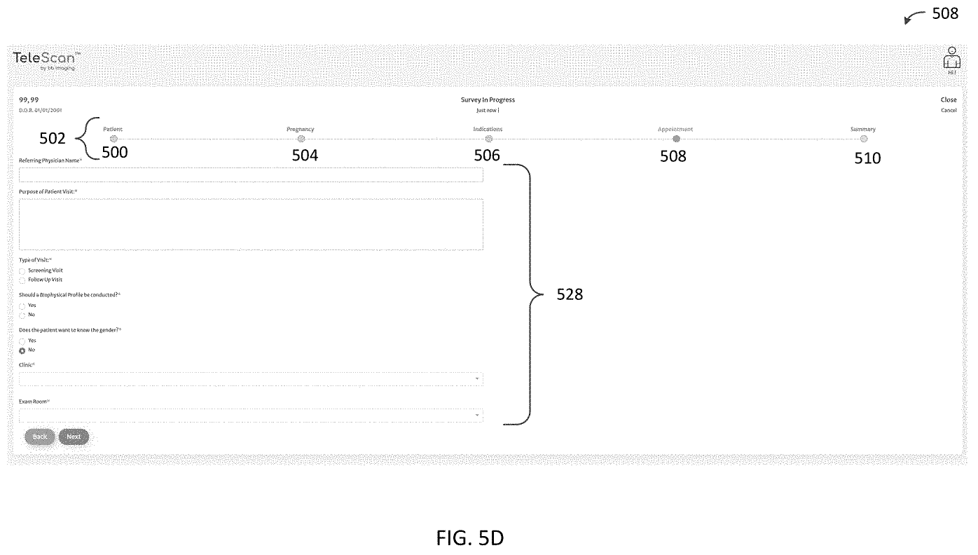

Now referring to FIG. 5A, a patient information screen 500 in accordance with one embodiment of the present disclosure is shown. The survey screen 500 includes a status bar 502 indicating respective screens in which different types of data are entered. For example, the patient information screen 500, pregnancy information screen 504, indications screen 506, appointment screen 508 and summary information screen 510. More or less screens can be used. Other screens may include general information screen, vitals screen, medical history screen, referring physician information screen, examination information screen, etc. The detailed patient information 512 is shown below the status bar 502 and includes the patient's last name, first name, ID, date of birth, maiden name, zip code, phone number, email address, height, weight, and blood pressure. The patient information 512 is not limited to the information shown.

Now referring to FIG. 5B, a patient pregnancy information screen 504 in accordance with one embodiment of the present disclosure is shown. The survey screen 500 includes a status bar 502 indicating respective screens in which different types of data are entered. For example, the patient information screen 500, pregnancy information screen 504, indications screen 506, appointment screen 508 and summary information screen 510. More or less screens can be used. The detailed pregnancy information 514 is shown below the status bar 502 and includes current pregnancy information 516 and pregnancy history 518. The current pregnancy information 516 includes “Have you had an ultrasound for this pregnancy?”, “Is this pregnancy the result of In Vitro Fertilization (IVG)?”, pre-pregnancy weight, number of gestations, “Is there fetal movement?”, “Prenatal Screening Test Result?”, and “Has the patient had a LEEP procedure?”. The pregnancy history 518 includes number of >37 week deliveries, number of <37 week deliveries, number of C-Sections, number of live births, and number of miscarriages/abortions. The pregnancy information 514 is not limited to the information shown.

Referring now to FIG. 5C, an indication screen 506 in accordance with one embodiment of the present disclosure is shown. The survey screen 500 includes a status bar 502 indicating respective screens in which different types of data are entered. For example, the patient information screen 500, pregnancy information screen 504, indications screen 506, appointment screen 508 and summary information screen 510. More or less screens can be used. The detailed indications information 520 is shown below the status bar 502 and includes current maternal information 522, fetal anomaly information 524 and other reported issues 526. The current maternal information 522 includes patient or immediate family history of cardiac defects, patient or immediate family history of chromosomal disorders, anxiety, depression, or seizure medication exposure (S SRI drugs, Lithium, Zoloft, etc. (abnormal screening, diabetes mellitus (Type I or II), gestational diabetes diagnosed prior to 24 weeks gestational age of current pregnancy, Lupus, +SSA/Ro antibodies, and unexplained polyhydramnios. Fetal anomaly information 524 includes echogenic intracardiac focus (EIF), two vessel umbilical cord, gastroschisis, omphalocele, cleft lip/palate, choroid plexus cysts, cardiac arrhythmia, thickened nuchal translucency/nuchal fold, hydrops, and pyelectasis. Other reported issues 526 include pain, bleeding, cramping and pelvic pressure. The indications information 520 is not limited to the information shown.

Now referring to FIG. 5D, an appointment screen 508 in accordance with one embodiment of the present disclosure is shown. The survey screen 500 includes a status bar 502 indicating respective screens in which different types of data are entered. For example, the patient information screen 500, pregnancy information screen 504, indications screen 506, appointment screen 508 and summary information screen 510. More or less screens can be used. The detailed appointment information 528 is shown below the status bar 502 and includes the referring physician name, purpose of patient visit, type of visit, “Should a Biophysical Profile be conducted?”, “Does the patient want to know the gender?”, clinic and exam room. The detailed appointment information 528 is not limited to the information shown.

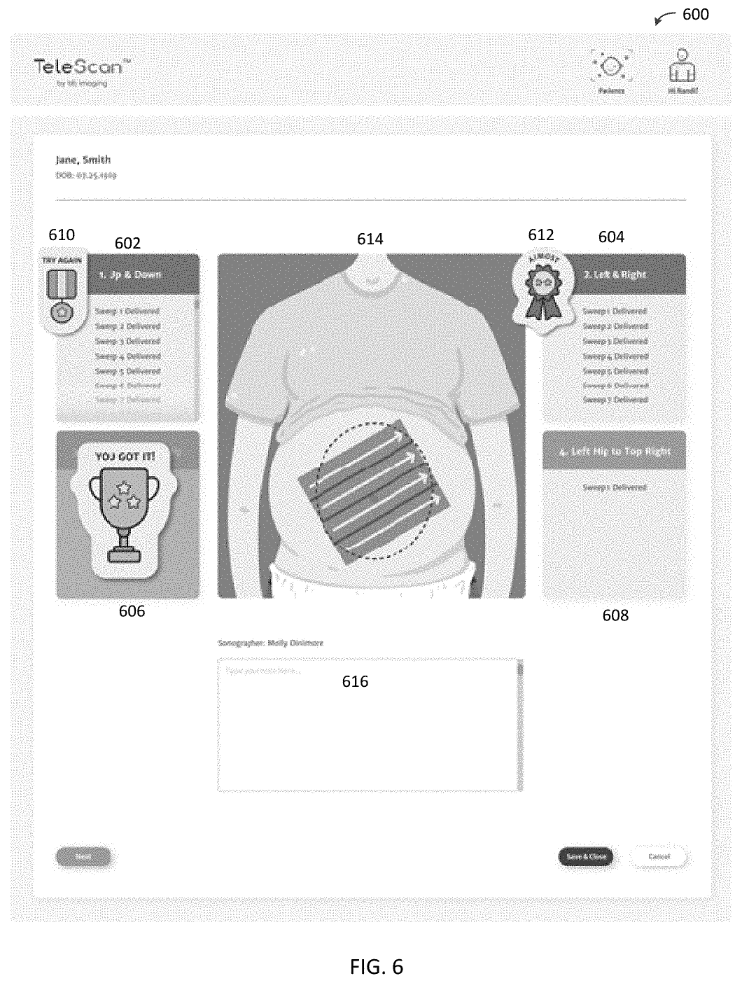

Referring now to FIG. 6, an instruction screen 600 with feedback from remote sonographer in accordance with one embodiment of the present disclosure is shown. The instruction screen 600 includes four windows 602, 604, 606 and 608 that group the sweeps by orientation. For example, window 602 shows the up and down sweeps, window 604 shows the left and right sweeps, window 606 shows the right hip to the top left sweeps, and window 608 shows the left hip to top right sweeps. These windows 602, 604, 606 and 608 include a status for the sweeps, such as “Delivered” in windows 602 and 604, or accepted “YOU GOT IT!” in window 606. If the sweeps are rejected, an indicator 610, such as “TRY AGAIN” is displayed. If the review of the sweeps is in process, an indicator 612, such as “ALMOST” is displayed. A middle window 614 shows a graphical depiction of what the current sweeps should be, such as arrows pointing from left hip to top right. A text box 616 for inputting notes is also provided.

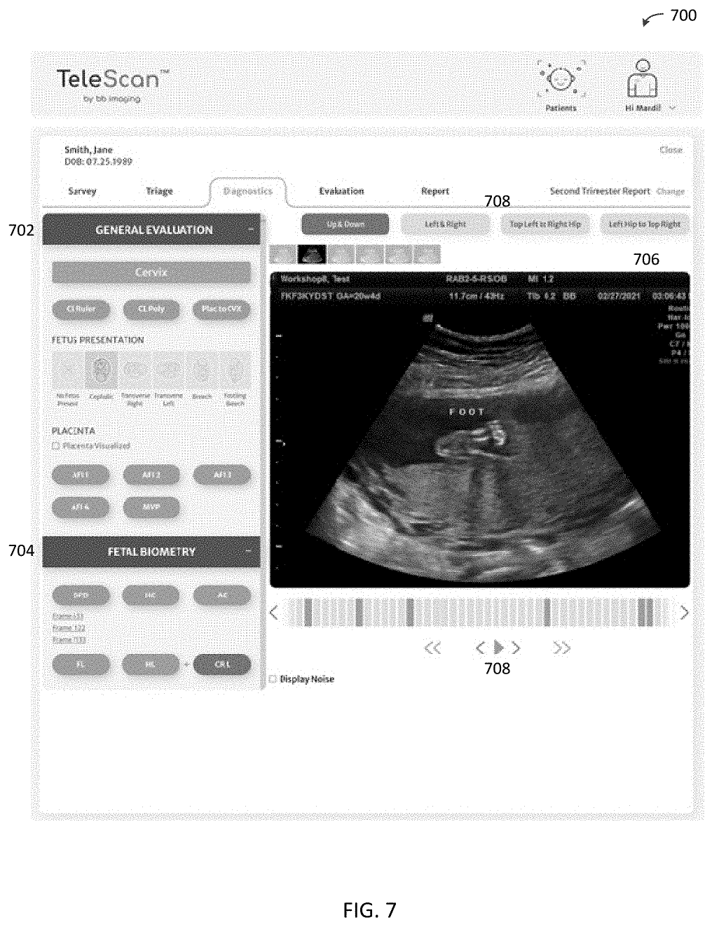

Now referring to FIG. 7, a remote sonographer diagnostics screen 700 in accordance with one embodiment of the present disclosure is shown. The sonographer diagnostics screen 700 includes a general evaluation section 702, a fetal biometry section 704, and a sonogram display screen 706. The sonogram sweeps are selected for display using buttons 708 (Up & Down, Left & Right, Top Left to Right Hip and Left Hip to Top Right). The viewing controls 708 allow specific images to be selected, play/pause, forward, fast forward, reverse, and fast reverse.

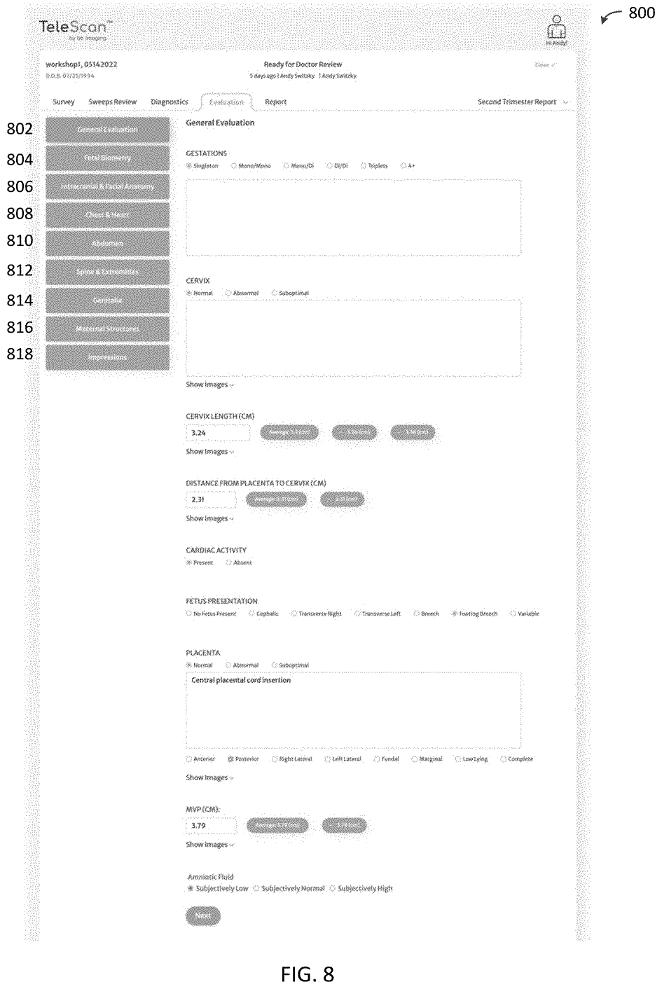

Referring now to FIG. 8, a sonographer diagnostic tools screen 800 in accordance with one embodiment of the present disclosure is shown. The sonographer diagnostic tools screen 800 provides multiple tool/report screens using button 802-818 (general evaluation 802, fetal biometry 804, intracranial and facial anatomy 806, chest and heart 808, abdomen 810, spine and extremities 812, genitailia 814, maternal structures 816 and impressions 818. More or less screens can be used. In this example, the general evaluation screen 802 is shown to the right of the buttons 802-818.

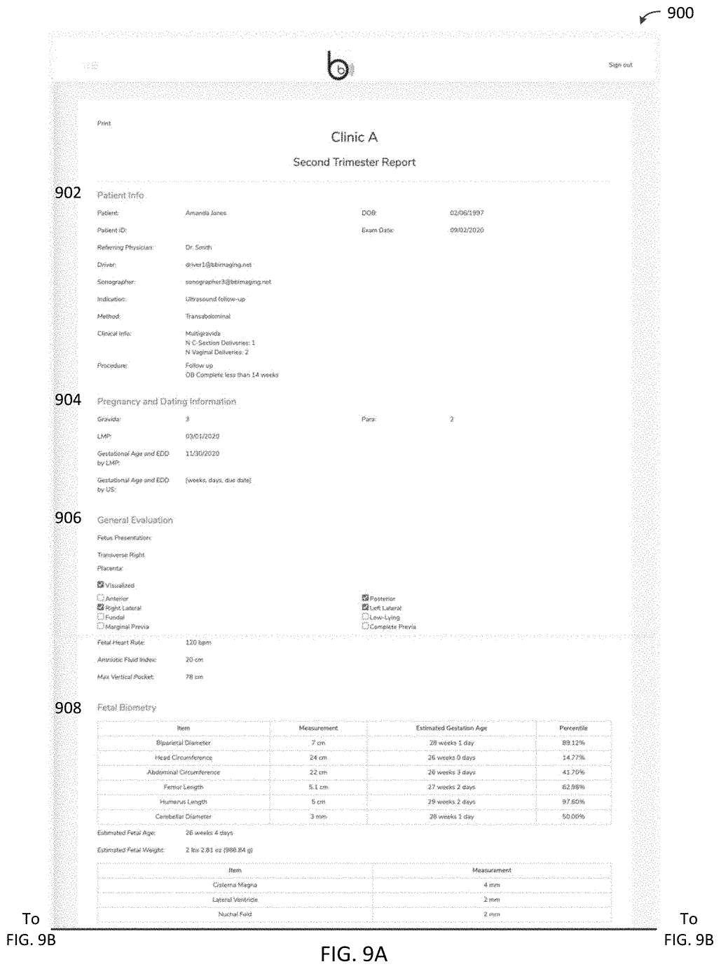

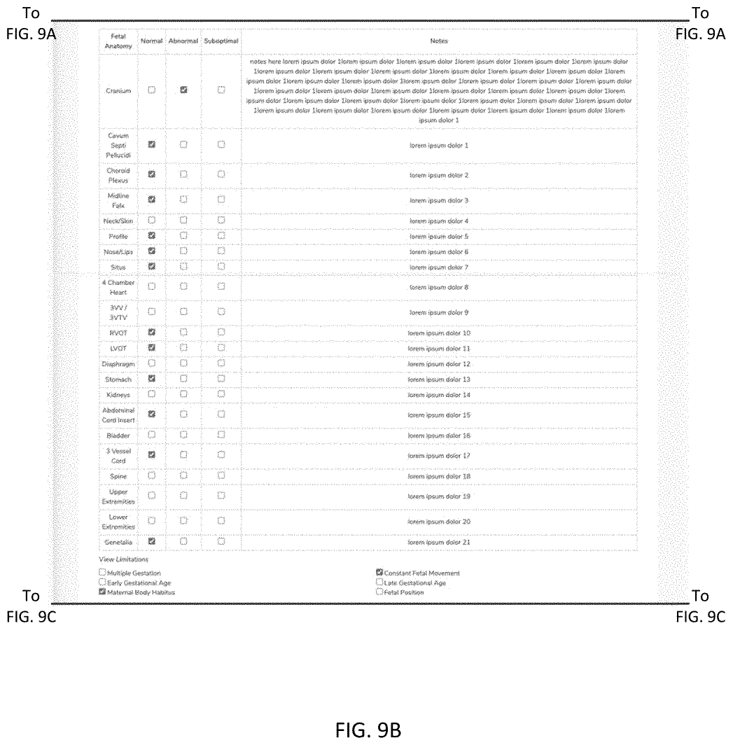

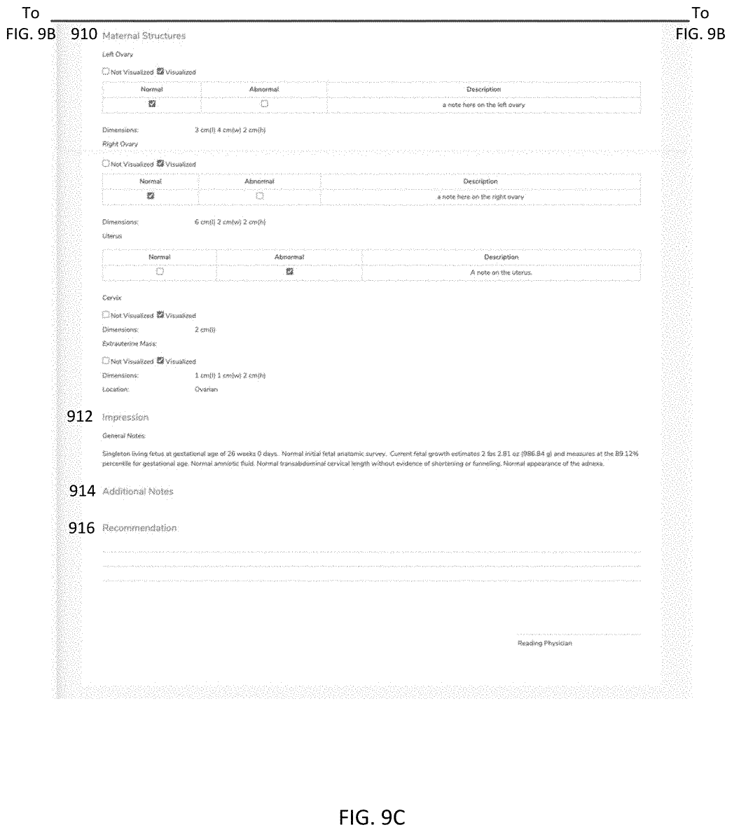

Now referring to FIGS. 9A-9C, a patient report 900 in accordance with one embodiment of the present disclosure is shown. The patient report 900 includes patient information 902, pregnancy and dating information 904, general evaluation 906, fetal biometry 908, maternal structures 910, impression 912, additional notes 914 and recommendation 916. More or less data can be used.

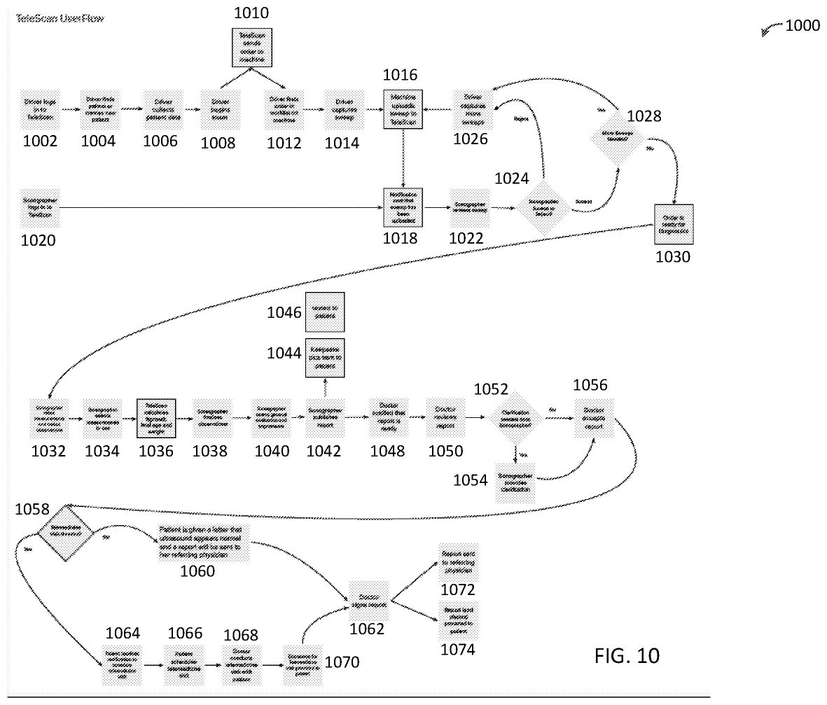

Referring now to FIG. 10, a flow chart of a method 1000 for obtaining and analyzing ultrasound data in accordance with another embodiment of the present disclosure is shown. A driver logs in to TeleScan™ in block 1002, finds patient or creates a new patient in block 1004, and collects patient data in block 1006. The driver begins the exam in block 1008. TeleScan™ sends the order to the ultrasound machine in block 1010. The driver finds the order in the worklist on the ultrasound machine in block 1012 and captures one or more sweeps in block 1014. The ultrasound machine uploads the one or more sweeps to TeleScan™ in block 1016, a notification is sent that the one or more sweeps have been unloaded in block 1018, and the sonographer logs in to TeleScan™ in block 1020. The sonographer reviews the one or more sweeps in block 1022. If the sonographer rejects the one or more sweeps, as determined in decision block 1024, the driver captures more sweeps in block 1016, and the upoad/review process repeats. If, however, the sonographer accepts the one or more sweeps, as determined in decision block 1024, and more sweeps are needed, as determined in decision block 1028, the driver captures more sweeps in block 1016, and the upoad/review process repeats. If, however, the sonographer accepts the one or more sweeps, as determined in decision block 1024, and no more sweeps are needed, as determined in decision block 1028, the order is ready for diagnostics in block 1030.

The sonographer takes measurements and observations in block 1032, and selects the measurements to use in block 1034. TeleScan™ calculates percent growth, fetal age and weight in block 1036. Sonographer finalizes observations in block 1038, enters general observations and impressions in block 1040, and publishes a report in block 1042. Keepsake pictures are sent to the patient in block 1044 or texted to the patient in block 1046. After the report is published in block 1042, the doctor is notified that the report is ready in block 1048, and the doctor reviews the report in block 1050. If clarification is needed from the sonographer, as determined in decision block 1052, the sonographer provides clarification in block 1054. Thereafter, or if no clarification is needed, as determined in decision block 1052, the doctor accepts the report in block 1056. If a telemedicine visit is not needed, as determined in decision block 1058, the patient is given a letter that the ultrasound appears normal and a report will be sent to the referring physician in block 1060, and the doctor signs the resport in block 1062. If a telemedicine visit is needed, as determined in decision block 1058, the patient receives notification to schedule a telemedicine visit in block 1064. The patient schedules the telemedicine visit in block 1066, and the doctor conducts the telemedicine visit with the patient in block 1068. A document for the telemedicine visit is provided to the patient in block 1070, and the doctor signs the report in block 1062. Thereafter, the report is sent to the referring physician in block 1072, and the report (and photo) are provided to the patient in block 1074.

Various embodiments of the present disclosure provide many of these features:

A. Cloud-Based

1. Biometric Measurements— OEM agnostic and operating system agnostic

2. Annotation process— PAX system developed to be used specifically for teleultrasound. The concept of “teleultrasound” is novel. Currently, unknowing users can't capture the images onsite while a skilled sonographer works remotely. This capability to work remotely now requires knowledgeable in-person staffers. Anatomical measurements must also be acquired in the ultrasound equipment for remote operability in the current systems. TeleScan allows for measurements and percentile calculations to be captured off-site. It also guides an unskilled healthcare worker to capture necessary anatomy “unknowingly.”

3. Cloud-Based Ultrasound Machine—TeleScan™ allows post-production annotation, manipulation, documentation, video communication, and text communication between users. TeleScan™ promotes the usage of remote ultrasound in all ultrasound modalities, including breast, cardiovascular, musculoskeletal, small parts, OB/GYN, and even veterinary scanning.

B. Sweep Pattern

Universal Users—Ultrasound systems require knowledgeable sonographers to utilize ultrasound equipment effectively, and TeleScan™ separates the need for a knowledgeable sonographer for successful report creation.

1. Image Acquisition Training— 4 Ps are custom development training created by BB Imaging. Position, Pace, Pressure, and Pattern.

2. Feedback Triage—immediate review by AI or human interaction provides remote “Driver” synchronous and asynchronous feedback if more images are required.

3. Speed of Exam—focusing on the sweep patterns allows for shortened exam length for moms

4. Patient-generated scans—this system allows for the future of ultrasound to be put in the hands of patients. TeleScan's roadmap splits the capability of a sonographer physically performing the scan and the capability of a sonographer remotely creating the diagnostic report.

C. Calculations

1. Cloud-based calculations completed outside of ultrasound machine.

2. Algorithms prompt potential pathologies for diagnostic support to physicians.

3. Percentiles are auto-calculated, TeleScan™ allows flexibility for selecting the preferred measurements and author in a cloud-based system.

D. Annotation

1. A unique system was implemented to complete the annotations on Digital Imaging and Communications in Medicine (DICOM) files. This annotation automatically transmits to the report.

2. Measurement tools, tagging, and prioritization are completed by expert sonographers using the custom tool developed in TeleScan™.

3. A “movie reel” prioritizes valuable images and puts them in sequential order for ease of diagnostic reporting.

4. Images populate into a report in order of importance and following industry standards for a unique, speedy documentation for healthcare providers.

E. Machine Learning/Artificial Intelligence

1. Machine Learning/Artificial Intelligence used in ultrasound image processing—TeleScan™ will identify images with the support of algorithms particular to image processing that will shorten reporting time. In essence, TeleScan™ will learn from existing annotated images to suggest annotations.

2. Algorithms utilized to support physicians in the potential diagnosis of rare pathology leveraging data collection.

F. Approach

1. Flattening of images for download and post-capture annotation simplifies documentation. DICOM viewer in the same application as the annotations is novel, allowing for seamless processing.

2. Approach to viewing—a movie reel approach was developed along with tagging system that allows for faster identification of “best” images.

3. Diagnostic Support—TeleScan™ will synthesize maternal medical history and demographics, reported lab values, common diagnoses associated with demographic and contextual information to suggest a deeper review of anatomical structures for better. This is novel and of particular importance.

4. TeleScan™ increases accessibility to ultrasound by removing the dependence on skilled users.

G. Sonographer Model

1. An arbitrage model of employment of sonographers.

2. Onboarding and training of sonographer.

3. Certification of “TeleScan-endorsed” sonographers.

H. NON FUNGIBLE TOKENS of persons first image/ultrasound presented to mom.

I. VIRTUAL REALITY/AUGMENTED REALITY of ultrasound images in development.

Circuits can be implemented with, but are not limited to, single or combinations of discrete electrical and electronic components, integrated circuits, semiconductor devices, analog devices, digital devices, etc. Elements can be coupled together using any type of suitable direct or indirect connection between the elements including, but not limited to, wires, pathways, channels, vias, electromagnetic induction, electrostatic charges, optical links, wireless communication links, etc.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains.

All publications and patent applications are herein incorporated by reference to the same extent as if each individual publication or patent application was specifically and individually indicated to be incorporated by reference.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms such as “above,” “below,” “upper,” “lower,” or other like terms to describe a spatial relationship between various components or to describe the spatial orientation of aspects of such components should be understood to describe a relative relationship between the components or a spatial orientation of aspects of such components, respectively, as the device described herein may be oriented in any desired direction.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of.” As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step, or a limitation) or group of integers (e.g., feature(s), element(s), characteristic(s), property(ies), method/process(s) steps, or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about,” “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the devices and/or methods disclosed and claimed herein can be made and executed without undue experimentation in light of the present disclosure. While the devices and/or methods of this invention have been described in terms of particular embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope, and concept of the invention as defined by the appended claims.

Furthermore, no limitations are intended to the details of construction or design herein shown, other than as described in the claims below. It is therefore evident that the particular embodiments disclosed above may be altered or modified and all such variations are considered within the scope and spirit of the disclosure. Accordingly, the protection sought herein is as set forth in the claims below.

Modifications, additions, or omissions may be made to the systems and apparatuses described herein without departing from the scope of the invention. The components of the systems and apparatuses may be integrated or separated. Moreover, the operations of the systems and apparatuses may be performed by more, fewer, or other components. The methods may include more, fewer, or other steps. Additionally, steps may be performed in any suitable order.

To aid the Patent Office, and any readers of any patent issued on this application in interpreting the claims appended hereto, applicants wish to note that they do not intend any of the appended claims to invoke 35 U.S.C. § 112(f) as it exists on the date of filing hereof unless the words “means for” or “step for” are explicitly used in the particular claim.

What is claimed is:

1. A computerized method for obtaining and analyzing ultrasound data, the method comprising:
creating by a computer, an electronic ultrasound order for a subject;
creating by a computer, an electronic worklist, and downloading the electronic worklist from the computer to an ultrasound machine, wherein the electronic worklist comprises instructions to conduct one or more sets of scanning sweeps, and the ultrasound machine is remotely located with respect to the computer;
receiving by the computer, one or more computer files containing the ultrasound data corresponding to the one or more sets of scanning sweeps from the ultrasound machine;
conducting by the computer, diagnostics and an evaluation for the subject using the set of scanning sweeps; and
creating by the computer, an electronic report for the subject using the diagnostics and the evaluation.

2. The method of claim 1, further comprising conducting the one or more sets of scanning sweeps using a transducer connected to the ultrasound machine.

3. The method of claim 2, wherein the one or more sets of scanning sweeps are conducted by a technician, the subject or an automated device.

4. The method of claim 1, wherein the one or more set scanning sweeps comprises:
a set of up and down scanning sweeps;
a set of left and right scanning sweeps;
a set of lower right to upper left scanning sweeps; or
a set of lower left to upper right scanning sweeps.

5. The method of claim 1, wherein the instructions comprise an electronic graphical representation of the one or more sets of scanning sweeps.

6. The method of claim 1, further comprising reviewing the set of scanning sweeps.

7. The method of claim 6, further comprising sending, by the computer, an acceptance or rejection of all or part of the set of scanning sweeps to a user device.

8. The method of claim 7, wherein the acceptance or rejection is provided in real time or near real time.

9. The method of claim 7, wherein the review, acceptance or rejection is performed by an artificial intelligence or machine learning program running on the computer.

10. The method of claim 7, wherein the rejection include one or more instructions on how to overcome the rejection.

11. The method of claim 7, further comprising:
receiving by the computer, one or more computer files containing a new set of scanning sweeps corresponding the rejected scanning sweeps;
reviewing by the computer, the new set of scanning sweeps; and
sending by the computer, an acceptance or rejection of all or part of the new set of scanning sweeps to user device.

12. The method of claim 1, further comprising providing by the computer, an electronic diagnostic tool comprising a set of diagnostic data selections and an interactive display of the one or more sets of scanning sweeps.

13. The method of claim 1, further comprising performing by the computer, one or more measurements or percentile calculations.

14. The method of claim 13, wherein the one or more measurements or percentile calculations are automatically performed by the computer.

15. The method of claim 1, further comprising providing by the computer, a diagnosis based on the diagnostics, the evaluation, a patient survey or a percentile calculations using an artificial intelligence or machine learning program running on the computer.

16. The method of claim 1, further comprising annotating by the computer, the one or more computer files.

17. The method of claim 1, further comprising prioritizing by the computer, a set of images corresponding to the one or more sets of scanning sweeps.

18. The method of claim 17, further comprising incorporating by the computer, the prioritized set of images into the electronic report.

19. The method of claim 1, further comprising creating by the computer, a virtual reality or augmented reality of one or more images corresponding to the one or more sets of scanning sweeps.

20. The method of claim 1, further comprising creating by the computer, a non fungible token of one or more images corresponding to the one or more sets of scans.

21. The method of claim 1, further comprising providing by the computer, an electronic report generation tool comprising a report template and a set of reporting selections corresponding to one or more portions of the subject.

22. The method of claim 1, further comprising sending by the computer, the electronic report to one or more electronic devices corresponding to the subject, a medical professional or both.

23. The method of claim 1, further comprising establishing by the computer, a communications link between the computer and the ultrasound machine.

24. A system for obtaining and analyzing ultrasound data, the system comprising:
an ultrasound machine;
one or more user devices; and
a computer communicably coupled to the ultrasound machine and the one or more user devices, wherein the computer is remotely located with respect to the ultrasound machine and the computer is configured to: create an electronic ultrasound order for a subject, create and download an electronic worklist to the ultrasound machine, wherein the electronic worklist comprises instructions to conduct one or more sets of scanning sweeps, receive one or more computer files containing the ultrasound data comprising the one or more sets of scanning sweeps from the ultrasound machine, conduct diagnostics and an evaluation for the subject using the set of scanning sweeps, and create an electronic report for the subject using the diagnostics and the evaluation.

25. The system of claim 24, wherein the one or more sets of scanning sweeps are conducted by a technician, the subject or an automated device.

26. The system of claim 24, wherein the one or more set scanning sweeps comprises:
a set of up and down scanning sweeps;
a set of left and right scanning sweeps;
a set of lower left to upper right scanning sweeps; or
a set of lower left to upper right scanning sweeps.

27. The system of claim 24, wherein the instructions comprise an electronic graphical representation of the one or more sets of scanning sweeps.

28. The system of claim 24, wherein the computer is further configured to review the set of scanning sweeps.

29. The system of claim 28, wherein the computer is further configured to send an acceptance or rejection of all or part of the set of scanning sweeps to the computer.

30. The system of claim 29, wherein the acceptance or rejection is provided in real time or near real time.

31. The system of claim 29, wherein the review, acceptance or rejection is performed by an artificial intelligence or machine learning program running on the computer.

32. The system of claim 29, wherein the rejection include one or more instructions on how to overcome the rejection.

33. The system of claim 29, wherein the computer is further configured to:
receive one or more computer files containing a new set of scanning sweeps corresponding the rejected scanning sweeps;
review the new set of scanning sweeps; and
send an acceptance or rejection of all or part of the new set of scanning sweeps to the ultrasound machine.

34. The system of claim 24, wherein the computer is further configured to provide an electronic diagnostic tool comprising a set of diagnostic data selections and an interactive display of the one or more sets of scanning sweeps.

35. The system of claim 24, wherein the computer is further configured to perform one or more measurements or percentile calculations.

36. The system of claim 35, wherein the one or more measurements or percentile calculations are automatically performed by the computer.

37. The system of claim 24, wherein the computer is further configured to provide a diagnosis based on the diagnostics, the evaluation, a patient survey or a percentile calculations using an artificial intelligence or machine learning program running on the computer.

38. The system of claim 24, wherein the computer is further configured to annotate the one or more computer files.

39. The system of claim 24, wherein the computer is further configured to prioritize a set of images corresponding to the one or more sets of scanning sweeps.

40. The system of claim 39, wherein the computer is further configured to incorporate the prioritized set of images into the electronic report.

41. The system of claim 24, wherein the computer is further configured to create a virtual reality or augmented reality of one or more images corresponding to the one or more sets of scanning sweeps.

42. The system of claim 24, wherein the computer is further configured to create a non fungible token of one or more images corresponding to the one or more sets of scans.

43. The system of claim 24, wherein the computer is further configured to provide an electronic report generation tool comprising a report template and a set of reporting selections corresponding to one or more portions of the subject.

44. The system of claim 24, wherein the computer is further configured to send the electronic report to one or more electronic devices corresponding to the subject, a medical professional or both.
